(19) United States
(12) Patent Application Publication
(10) Pub. No.: US 2020/0229522 A1
(43) Pub. Date: Jul. 23, 2020

(54) MEDICAL GLOVE ANTI-SLIP AND FLUID GUARD BAND

(71) Applicant: Garry Kitay, Jacksonville, FL (US)

(72) Inventor: Garry Kitay, Jacksonville, FL (US)

(21) Appl. No.: 16/745,319

(22) Filed: Jan. 16, 2020

Related U.S. Application Data
(60) Provisional application No. 62/793,525, filed on Jan. 17, 2019.

(51) Int. Cl. A41D 19/00; A61L 31/10

(52) U.S. Cl. CPC A41D 19/0089; A61B 42/10; A61L 31/10

(57) ABSTRACT

An elastic band for attachment over the juncture of a surgical glove and sleeve of a surgical gown is disclosed. The elastic band has an abrasive inner surface in contact with the juncture to limit movement of the band relative to the juncture. A circumferential portion of the inner surface is concave. The band may be of variable circumferences to fit the user, adjusting to the size of the user's wrist.

### BACKGROUND OF THE INVENTION

This product is directed to the common problem of medical gloves, including the sterile surgical variety, from sliding, slipping or peeling down the sleeve toward the wrist from its original positon on the forearm. This can aid in preventing bacterial contamination of the glove-gown interface, thereby reducing the incidence of surgical site infections. It also aids in preventing fluid seeping beneath the glove and making contact with the healthcare professional's skin. Both these events, glove sliding and skin exposure to patient fluids and tissue, elevate the risk of contamination both for the patient and health care provider. Minimizing this risk is a medical priority.

Standard elastic bands have been used by doctors and other healthcare professionals to suboptimally limit liquids from coming into contact with their hand and forearm skin. The standard bands would be placed around the medical gloves to protect the wrist portion of the medical gloves. Also, there are specialized bands for use with medical gloves, such as in U.S. Pat. No. 6,941,579 (“'579 Patent”). In FIGS. 17 and 18 of the '579 patent there is shown a device referred to as a bead for use with a gown and gloves that is disclosed as having the potential to be a separately applied elastic band. Various configurations of the bead or band are shown in FIG. 18 of the '579 patent. The bead or band of the '579 patent does not reveal the inner surface being abrasive or adhesive, but rather only shows a smooth inner surface. Also, the inner surface of the bead is flat, and does not have a concave segment to fit over and cover the top cuff of a glove.

### OBJECTS OF THE INVENTION

An object of the invention is to provide a band that contributes to a liquid or fluid resistant seal that prevents fluids or particles from passing through the interface of a glove and sleeve. In this manner, a continuous seal is created, thereby, preventing glove slippage, and compressing the sleeve channels that would otherwise potentially allow fluids, formites or bacteria, that normally inhabit the skin surfaces of the hands of healthcare workers, from passing through the interface.

Another object of the invention is to provide a sleeve and glove barrier that will not slip.

A further object of the invention is that it is easy to use.

A further object of the invention is that it is inexpensive to make.

### SUMMARY OF THE INVENTION

An elastic band for placement over the juncture of a surgical glove and sleeve of a surgical gown is disclosed. The elastic band has a roughened or abrasive inner surface in contact with the juncture to limit movement of the band relative to the juncture. A circumferential portion of the inner surface is concave. The band may be of variable circumferences to fit the user, adjusting to the size of the user's wrist. The band may be a continuous elastic ring, or it may be discontinuous and connected at its ends with a hook and loop fastener or other coupling device.

### DESCRIPTION OF THE DRAWINGS

The present invention and its advantages may be understood by reference to the detailed description section when read with the accompanying drawings briefly described below.

FIG. 1 shows a top perspective view of the band of the present invention.

FIG. 2A shows a user wearing a glove and gown without the band.

FIG. 2B shows the user wearing a glove and gown with the band.

FIG. 3 shows a cross section of the band.

### DETAILED DESCRIPTION

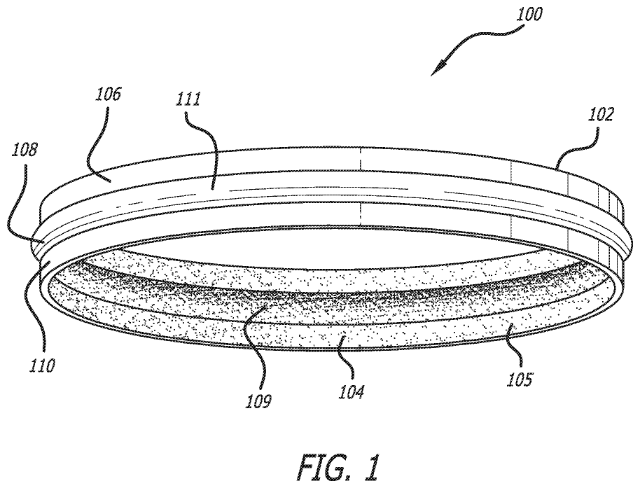

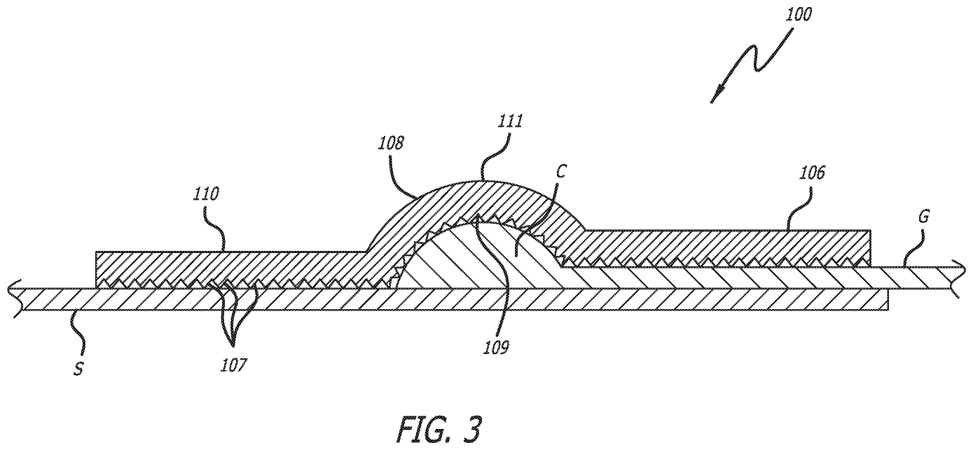

Referring to FIGS. 1 and 3, the band 100 of the present invention is made of a wide flexible elastic material having an outer portion 102 and an inner portion 104. The outer portion 102 of the band has three segments 106, 108, and 110. The middle segment 108 has a convex outer portion 111 and a concave inner portion 109 to cover the top cuff of a glove. In the preferred embodiment, the inner portion 104 is made of a non-slip, roughened portion 105 as shown in FIG. 1, or corrugated or ridged portion 107 as shown in FIG. 3, to prevent it from shifting during use. It is also possible to have adhesive 104 covering at least a part of the inner portion 104. While the three segments 106, 108 and 110 are illustrated as having the same width, the widths can be different. For example, segment 106 can be wider than segment 110 to make it easier to grab the band for placement or removal.

The band made of an elastic material consisting of a closed loop having a diameter in its lax form smaller than the circumference of the forearm of the user and stretchable so as to permit the band to fit over the hand and forearm of the user and to restrict liquid from accessing the interior of the glove and prevent particles migrating from the hands to the sleeves.

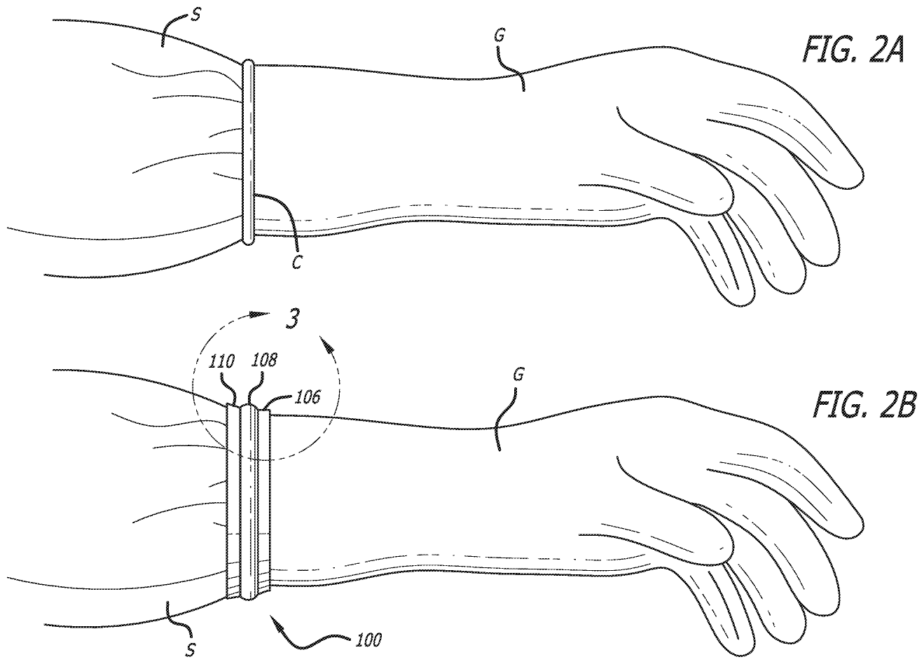

FIG. 2A shows a glove (G) and sleeve (S) without the band 100. FIG. 2B shows the glove (G) and sleeve (S) with the band 100 in place, with the concave inner portion 109 fitting over the top cuff (C) of the glove (G). The band 100 compresses the opening of the glove (G), achieving the first goal of immobilizing the cuff of the glove and preventing it from slipping. Second, the band prevents fluids from entering the glove (G), so that the medical provider is better protected from exposure to the patient's tissues and fluids which at times carries risk of bacterial and viral contamination to the treating healthcare professional, and conversely, the patient is protected from contamination by the health worker.

In the preferred embodiment the band 100 is latex free and appropriate for medical use in multiple settings. They can be packaged in pairs for sterile use, such as in the operating room setting with surgical gloves. They can also be used with nonsterile gloves, in settings like the emergency room and dental office. While one size fits most wrists, smaller and larger circumferences can be provided for smaller and larger wrists. The components of the invention described above may be made by processes known in the art such as extruding, molding dipping, or combinations thereof. The band may be a continuous elastic ring, or it may be discontinuous and connected at its ends with a hook and loop fastener or other coupling device.

The band 100 is used as follows: after scrubbing, the user dons the surgical gown, with the hands of the surgeon extending outside of the sleeves (S) of the gown. An assistant opens the glove (G) cuff (C) opening, and the user then pushes his or her hand into the fingers of the glove (G). The assistant nurse would then pull the cuff (C) of the glove (G) proximally toward the forearm over the baggy gown sleeve (S). The nurse would then stretch the band 100 over the hand of the surgeon and fit the band 100 over the top portion of the glove, with the middle segment 108 of the band 100 covering the cuff (C) of the glove (G). In this manner, the glove (G) is now firmly secured to the sleeve (S). The surgeon or health care provider can also don the gloves and band independently.

In the preferred embodiment the inner diameter of the band is between 1 3/4 inches to 2 ½ inches. The bands can be made in various sizes depending on the size of the forearm of the user. The width of the band in the preferred embodiment is approximately ¾ inches, but can have wider or narrower dimensions depending on the preferences of the user. This permits the band to maintain its shape without collapsing on itself as could occur if the band was much smaller. The three segments 106, 108 and 110 are all approximately ¼ inch inside as noted above, the width of the three segments 106, 108 and 110 can be varied. The thickness of the band in the preferred embodiment is 0.05 inches. The convex outer central segment 111 projects outwardly by approximately ⅛ inches and the concave inner segment 109 extends inwardly by approximately ⅛ inches. The dimension of the concave inner segment 109 can be sized so as to fit over the specific cuff of the glove (G) being used. While the outer portion 102 is shown with a convex segment 109, the outer portion 102 can be flat, curved, or any other desired shape.

While preferred embodiments have been shown, it will be understood that the invention is capable of numerous modifications, rearrangements, and substitution of parts without departing from the uniqueness of this invention as set forth in the claims section of this application.

1. A band used with a medical glove consisting of: A band made of
an elastic material consisting of a closed loop having a diameter in its lax form smaller than the circumference of the forearm of the user and stretchable so as to permit the band to fit over the hand and forearm of the user and resistant to glove slippage and liquid from having access to the interior of the glove;
an outer surface and an inner surface, the outer surface having an irregular surface and the inner surface having at least a portion thereof being roughened.

2. The band of claim 1 in which the outer surface has at least a portion being convex.

3. The band of claim 1 in which the band has a circumferential concave portion.

4. The band of claim 1 in which the inner surface has at least a part covered in adhesive.

5. A band used with a medical glove; said band consisting of a closed loop of an elastic material, the closed loop having a diameter in its lax form smaller than the circumference of the forearm of the user and stretchable so as to permit the band to fit over the hand and forearm of the user, and an outer surface and an inner surface, which has a circumferential concave portion.

6. The band of claim 5 in which at least a portion of the inner surface is roughened.

7. The band of claim 5 in which the outer surface has at least a portion being convex.

8. The band of claim 5 in which the inner surface has at least a portion covered in adhesive.

9. The band of claim 1 in which there are three segments, outer segments and a middle segment, the middle segment being concave.

10. The band of claim 1 in which the outer segments are of different widths.
